(19) United States
(12) Patent Application Publication
(10) Pub. No.: US 2017/0266029 A1
(43) Pub. Date: Sep. 21, 2017

(54) KIT AND METHOD FOR MAKING A WOUND PROTECTION DEVICE

(71) Applicant: MakeltMarketUSA.com, LLC., Charlotte, NC (US)

(72) Inventor: Katherine Liddle, Dunedin, FL (US)

(21) Appl. No.: 15/615,205

(22) Filed: Jun. 6, 2017

Related U.S. Application Data
(63) Continuation-in-part of application No. 14/470,276, filed on Aug. 27, 2014.
(60) Provisional application No. 62/021,442, filed on Jul. 7, 2014.
(60) Provisional application No. 61/995,225, filed on Apr. 7, 2014.

(51) Int. Cl. A61F 5/058

(52) U.S. Cl. CPC A61F 5/05841; A61F 5/058

(57) ABSTRACT

A kit and method are presented for making a mobile injury protection device, such as a splint, for protecting an injured body part, such as a limb. The kit includes a formable object and a heating source. The heating source is used to selectively heat the formable object to an elevated temperature, molding the formable object into a required shape for the injury protection device and allowing the molded object to cool to room temperature and become hard again. The formable object may be molded on or around and be in contact with the desired body part. The heating source may include a selectively activated chemical composition, for example by adding water or air to it.

### RELATED APPLICATIONS

This application claims priority to U.S. Provisional Patent Applications Nos. 61/995,225 filed Apr. 7, 2014, and 62/021,442 filed on Jul. 7, 2014, as well as U.S. non-provisional patent application Ser. No. 14/470,276, filed Aug. 27, 2014 and now pending, all of which are incorporated herein by reference in their entireties.

### BACKGROUND OF THE INVENTION

This invention pertains to a kit and associated method for mending or strengthening a patient's limb and, in particular, to a kit and method for making a device to aid in protecting, for example, an injured limb such as a finger, an arm, a leg of a person or of an animal. The kit includes a portable unit with a material that is hard and rigid at room temperature but soft and malleable at higher temperatures. The kit includes the material and a heating source for heating the material, and potentially other items needed for heating.

Description of the Prior Art

There are many splinting, strengthening, and repair systems available today. For example, instructions for addressing the problem of protecting or stabilizing a broken limb, especially in the field, or other environments where there is no immediate medical (or veterinary) assistance available, include finding sticks, twigs or other stiffening rod-shaped elements, positioning these elements adjacent to the broken limb (preferably on opposite sides thereof) and then tying them tightly together thereby immobilizing the limb. By immobilizing the area, the body is afforded an improved opportunity to heal itself by generating new tissues to fuse the broken ends of the bones together. If the process is done correctly, the limb will heal properly. If it is done incorrectly, the broken sections may grow together in an improper position, and surgical and/or other procedures must be used to reset the bone.

There are kits and methods available for use by professionals and technicians which comprise materials and instructions for splinting or strengthening or repair systems to be used in temporary or emergency situations to manage fractures or other injuries, including fractures or injuries to the jaw areas of humans and animals and to manage repairs for objects. Important factors for consideration in choosing a splinting material and method include: 1) a medical or other professional user may not be readily available, 2) the patient or non-professional user may or may not be required to form the device on their own, 3) forming the splint, strengthening, or repair device by a patient or non-professional user on their own may require activities which the patient or non-professional user may or may not be able to achieve, 4) the patient or user or object may be subject to further injury or damage if immediate splinting or strengthening or repair is not accomplished, and 5) the patient or a non-professional user or consumer may not be capable of following the kit and method instructions correctly and in a timely manner.

Therefore it would be beneficial to have a kit, with rapidly available resources, for forming a splint or strengthening or repair system, such as one which could be used in emergency situations, which is adaptable for forming an on-demand custom stabilizing or protective device.

### SUMMARY OF THE INVENTION

A goal of this invention is to create a kit and method of using the same for making an injury protection device, such as a splint, adapted to enable stabilization of the limb of a person or animal. The kit preferably includes instructions which are intuitive and easy to understand for both professional and non-professional users alike. Intuitive or easy to understand instructions allow the user to focus on creating a device of this invention well suited for its intended purpose.

In one embodiment, the present invention pertains to a kit including a formable object and a heating source. The heating source may be a chemically activated source that is selectively activated on demand to warm the formable material. The heating source may be, for example, one or more of a flameless heating compound or mixture of compounds that is activated on contact with air, water, mechanical manipulation, and so on.

The formable object is formed of a material that has a hard, rigid shape at room temperature and above (up to a threshold temperature range but may be soft and malleable at higher than threshold temperature). In its packaged state within the kit of the present invention, the object may be in any number of structured forms, such as in a ball or in a sheet. As a sheet, it may be packaged in a rolled form. As the material is heated above the threshold temperature range, it becomes malleable. As the material is heated above this temperature, it softens so that it can be molded into various shapes. The material maintains its molded shape after it cools to room temperature.

After it is softened, the formable material is shaped to form a splint or similar reinforcing or protective structure for the limb of a patient or animal. Optionally, wrapping tape may be added on top of the splint or between the splint and the patient's tissues.

Easy to read instructions are also included for using the kit to make a protective device.

### DESCRIPTION OF THE FIGURES

FIG. 1 shows the kit of the present invention and its components for making a portable wound management or protection device such as a shield for a burn or tissue deficit injury in accordance with this invention, together with some devices made using the kit.

FIG. 1A is a flow chart of the method used to make the burn protection device of FIG. 1;

FIG. 2 shows pieces of the formable material being positioned for wrapping;

FIG. 3 shows the bag used as one possible heating source for heating the formable and stretchable material;

FIG. 4 shows the bag with the formable material being heated;

FIG. 5 shows the wrapped formable material having been just removed from the flameless heater bag;

FIG. 6 shows the unwrapped formable material which is now ready to be formed as desired;

FIG. 7 shows the formable material being shaped;

FIG. 8 shows a shield, preferably used for a burn or another injury, on a hand using the formable material of FIG. 7; material being formed more completely and being used as a temporary or emergency burn protection and injury management device for a burned hand and fingers

FIG. 9 shows the underside of the finger of FIG. 8 that is not covered by material.

### DETAILED DESCRIPTION OF THE INVENTION

This invention is intended to provide an easy to use kit and method for wound management or protection or other similar repair or strengthening device or a splinting device for a human or animal or object. While referred to herein as a “splint” or in alternative terminology such as “wound covering” or “burn protecting device”, the present invention includes and is directed to a material which can be formed to cover a wound or immobilize a portion of a person or animal rapidly and without adverse effect.

A kit for making the above described device comprises a formable and stretchable material of chemistry, color, size and shape and thickness to suit particular or individual applications. The kit also includes a portable heating device or some other device for facilitating formation of the protective device for the immediate purpose—be it to cover a wound, a joint, or some other body portion—with the formable and stretchable material so that wound management or protection may take place at the time of injury even if physician or hospital care is not immediately accessible. Different chemistries, colors, sizes, shapes and thicknesses as well as different heating methods for the kit may be required depending on intended use by multiple industries: military, hospital, veterinary, dental, surgical, sports using the invention and as well on whether the invention is required to be used in an enclosed or open environment or whether water or air or another heating method is available at the point of injury or whether the invention is to be used within the U.S. or in other countries. The kit also includes instructions for making said device.

In one embodiment, the present invention pertains to a kit including at least a formable and stretchable material and a heating source. The heating source may be a chemically activated source that is selectively activated to warm the formable material. The heating source may be for example a flameless heating compound or mixture of compounds that is activated on contact with air, water, mechanical manipulation, and so on.

In one embodiment, the heating element, such as a chemical composition, is embedded in the material and is activated by water. In this embodiment, the kit may include a pack in which sterile water is included for mixing with the material, which itself may be within a sterile environment in the kit.

The terms “formable” and “stretchable” relative to material is used herein to define a material that has a solid, non-malleable shape at room temperature and somewhat above (up to a threshold temperature). The threshold temperature may be, for example, about 37°±5° C. As the material is heated above this temperature, it becomes soft and malleable so that it can be molded and stretched and sutured into various shapes. For example, the formable material may become sufficiently malleable so that it can be shaped and sutured as desired in the range of 45-75° C.

The term “wound management or protection device” is used to define a hard, rigid device at room temperature that can or has been applied to a portion of the body of the patient (either human and veterinary) to maintain that body portion stable and to protect and prevent its movement, for example, while a natural healing takes place.

The wound management or protection device could be a shield for a burn victim or a protection for a wound with deficit tissue and could be applied to injuries of a person or animal or to other body portions. The wound management or protection device can be applied to, over, or around the source of a puncture wound, such as a knife, so that the wound and instrument of puncture will remain stable during patient transport to physician or hospital care.

The formable and stretchable material may have several varied compositions. In one embodiment the compositions are biocompatible. For example, the formable material may be made of polycaprolactone, optionally with additives such as an anti-microbial agent or a colorant. The formable material may also be formed from other compounds, including compounds made from a polyvinylacetate or a thermoplastic material.

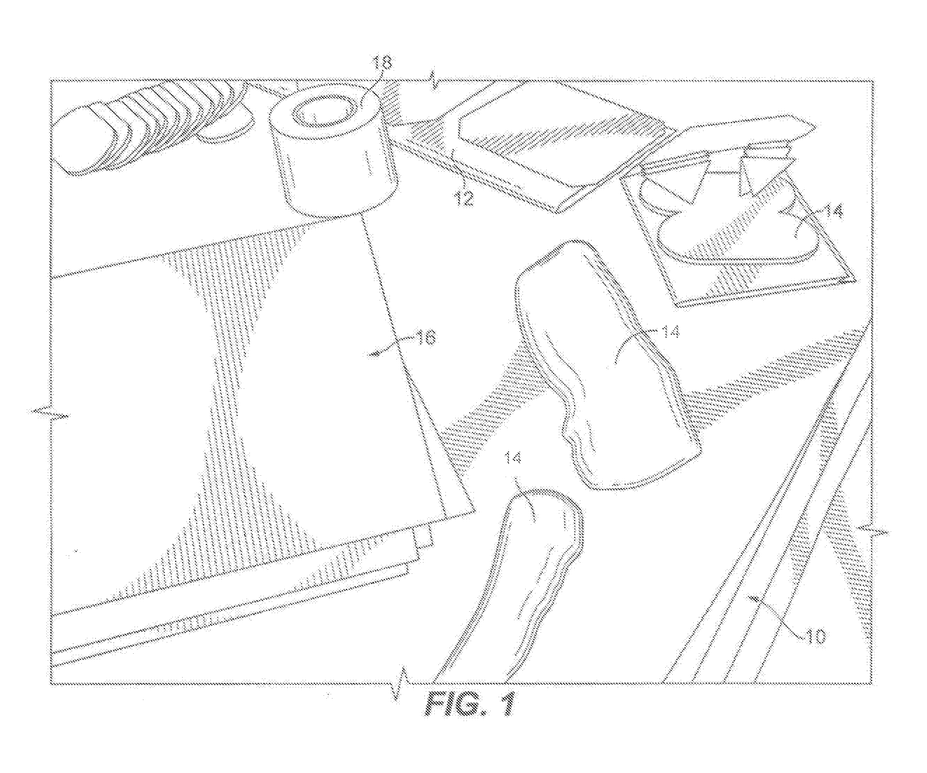

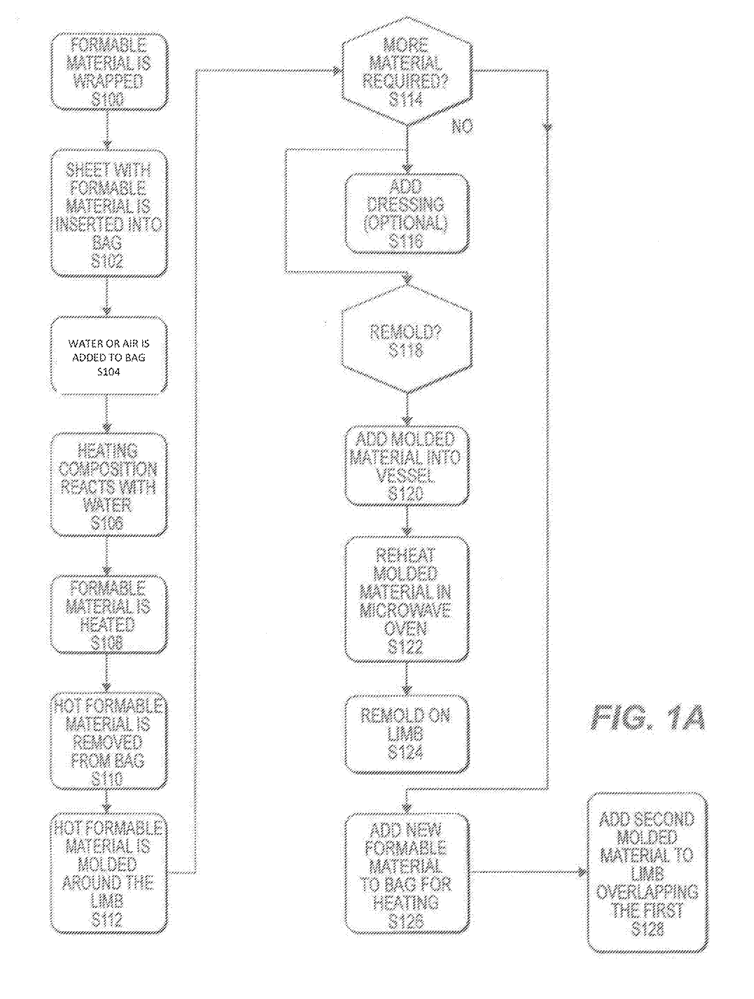

Referring to FIG. 1, a kit 10 constructed in accordance with this invention includes a bag or container 12 with the heating composition 12A (not shown), one or more pieces of formable material 14, one or more sheets of specially treated paper protective sheet 16 with sealing label, and, optionally a roll of tape 18 or other means of dressing a wound of a patient.

The container 12, as previously discussed, includes a heating composition, such as the ones described U.S. Pat. No. 5,517,981 to Taub. This composition heats when water is added to it. Other well-known heating compositions may be used as well.

The formable material 14 is provided in wafers or sheets sized and shaped for functionality in various wound protection devices. For example, the material can be provided in 4 in×4 in square pieces, or in pieces that have been cut-out so that they have a somewhat elliptical shape as shown. Each material wafer or sheet 14 may be greater or less than 2 millimeters thick. The kit itself and the contents may be sterile.

One application of the present invention is as an injury protection device. The device can be used, for example, on the palm of the hand. The material is substantially formable for the injury. The material is stretchable and can be directed, such as upwardly from the palm of the hand to separate burned fingers. The kit may also include an adherence, such as but not limited to tape or other adhering material, which can be used to hold the device in place, where the adherence is placed to surround the wound. The material used and its ability to be shaped consequential to being a part of the kit of the present invention permits this injury management to be accomplished on site at the point of injury even before transport to a medical facility or to the next level of care.

The material has the ability to be heated in the kit in a number of ways—water, air, hydrogen suppressed, inside the heating unit and wrapped to prevent adherence to the heating source. In addition, in a preferred embodiment the material is radiolucent so that medical personnel can determine the next step of care before removing the injury protective device from the victim.

That the material can be stretched thinly and wrapped or sutured while stretched is important.

The kit may further include gloves so as to retain sterility during application.

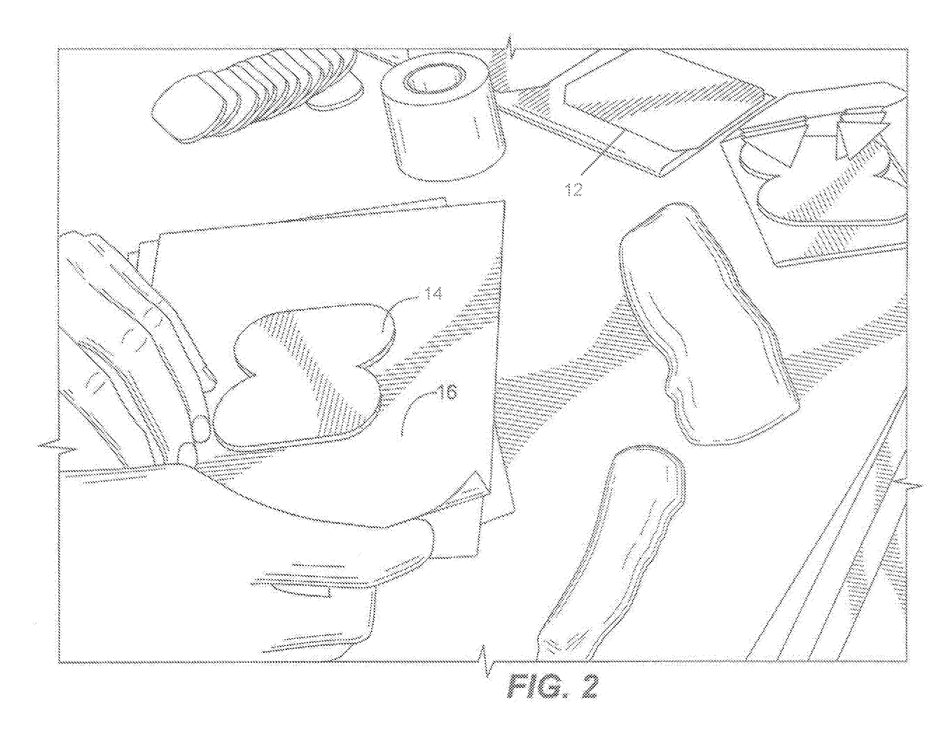

In one embodiment, the kit is used as illustrated in the Figures. First, the formable material 14 is placed on a protective sheet of particular qualities 16 and then wrapped with the sheet 16 (FIG. 2, step S100 FIG. 1A). The sheet 16 with the material 14 is then placed into bag 12 so that the sheet 16 is in intimate contact with the heating composition 12A (step S102). The sheet 16 is comprised of a thin film of plastic or a thin paper coated with a suitable substance that prevents the sheet 16 from sticking to the material 14 even in the presence of heat. The sheet 16 prevents direct contact between the heating composition 12A and the material 14. The heating composition 12A may be in the form of several components in a powdery or granulated form. A predetermined amount of water (e.g., a spoonful) is then added to the heating composition 12A in bag 12 (Step S104) starting a chemical reaction between the heating composition 12A and the water.

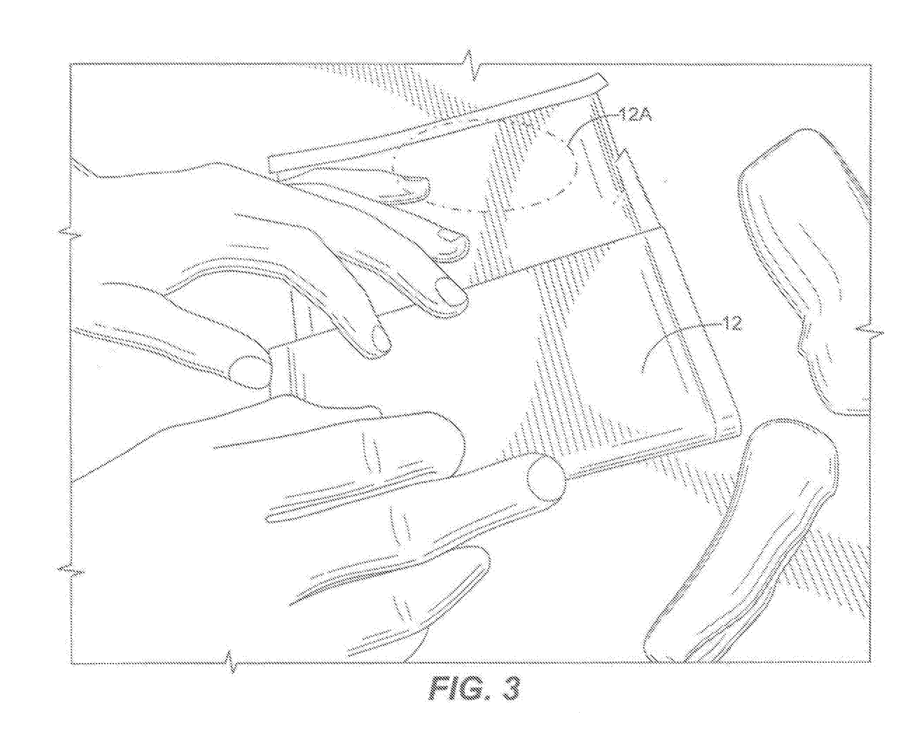

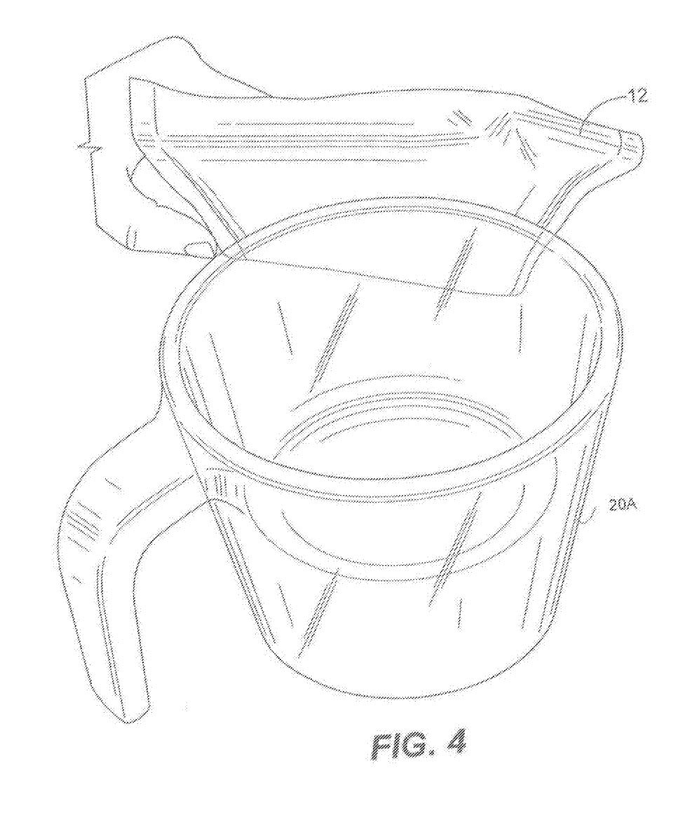

The bag 12 is then closed (FIG. 3), as specified in the instructions, sufficiently so that the heat generated by the heating composition heats the formable material. The closed bag is maintained in this condition for several minutes to allow the formable material to heat up and soften. (The inventor has found that typically two minutes is sufficient). Preferably during this waiting stage the container 12 is maintained in an upright position by leaning it against a cup of water 20A (FIG. 4). The heating composition 12A is in container 12. Depending upon the nature of the heating, formable material 14 may also be in container 12 or may be proximal to it. (Water from cup 20A may have been previously poured into the bag as described as the cup and water may be pre-sterilized). During this waiting time, the heating composition 12A reacts with the water and generates heat that heats up the material 14 to about 45-75° C. (steps S106, S108 in FIG. 1A).

In one embodiment of the pesent invention, an exothermic heater is included in a self-contained package which can be activated by removing a seal, thereby exposing the heating element to air. In one structure, the heater includes a packaged film layer, an air diffuser/waterproof layer, a heater, and a second film layer, although variations are possible. The heater is tunable to activate in a defined time, typically between 2 and 15 minutes, depending upon size, weight, and temperature of the product to be heated.

In another embodiment, the kit of the present invention includes hydrogen suppressed technology, such as, but not limited to, that described in Taub. The use of this type of technology permits use in particular environments, such as in an airplane or in a tent or other enclosed area. In this embodiment, the hydrogen component of the heating composition is controlled in such a way as to permit activation when water is added but without the toxic fumes emitted when the formula without hydrogen suppression is used in the heating composition in a closed area.

The kit of these embodiments enables the user to balance the energy input from the chemicals to the energy uptake from the water and the material 14 to predictably achieve its ideal formable temperature.

In another embodiment, the kit includes a customized heating container wherein a flap is removed from an outer portion of the container. Upon the removal of the flap, air penetrates remaining holes in the container to heat the contents. In this embodiment the energy (heat) is created from the reaction of zinc with air and results in no release of potentially toxic byproducts during the heating cycle. In this embodiment the user can set both the temperature of the material 14 and the time required to attain the target temperature.

The material becomes malleable at an elevated temperature as described herein. The elevated temperature is one at which the formable material is suitable to be placed on a human or animal body without any heat-insulating or other barrier material between the formable material and the human or animal body part. The formable material is heated by placing the formable material and the heating source in the container.

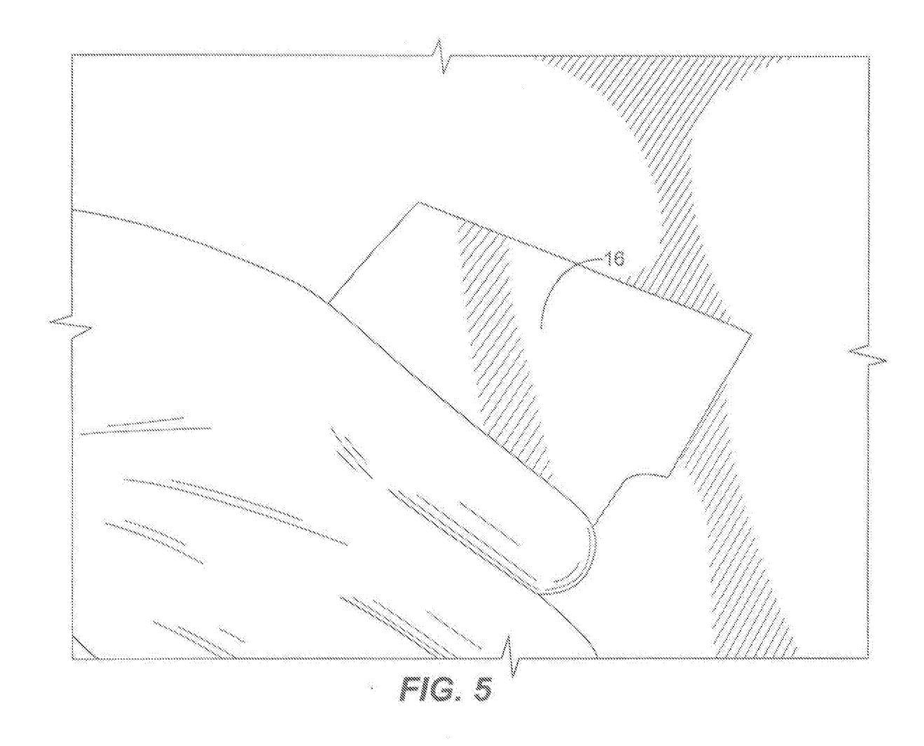

As the material 14 heats up, it becomes malleable so that it is easy to work it by hand and shape it into any desired shape. After the formable material is heated to the desired temperature, the sheet 16 with the soft formable material is removed from container 12 (FIG. 5) and the formable material 14 is separated from the specially treated sheet 16. Advantageously, at room temperature, the formable material is solid and opaque. As the formable material attains its malleable form, the formable material becomes increasingly translucent. Thus, the person making the injury protection device can easily determine whether the formable material 14 has been heated sufficiently.

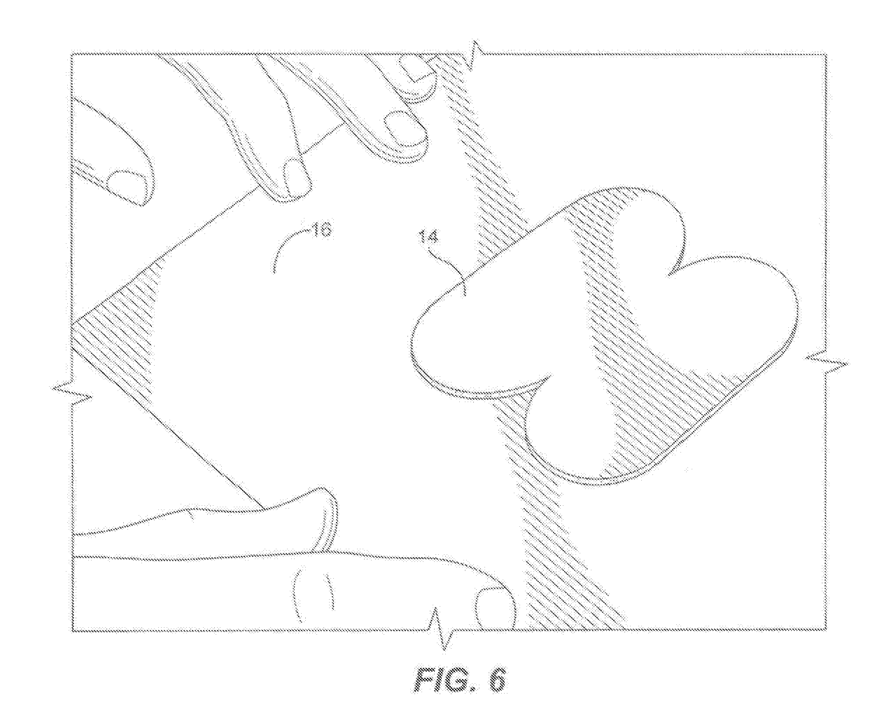

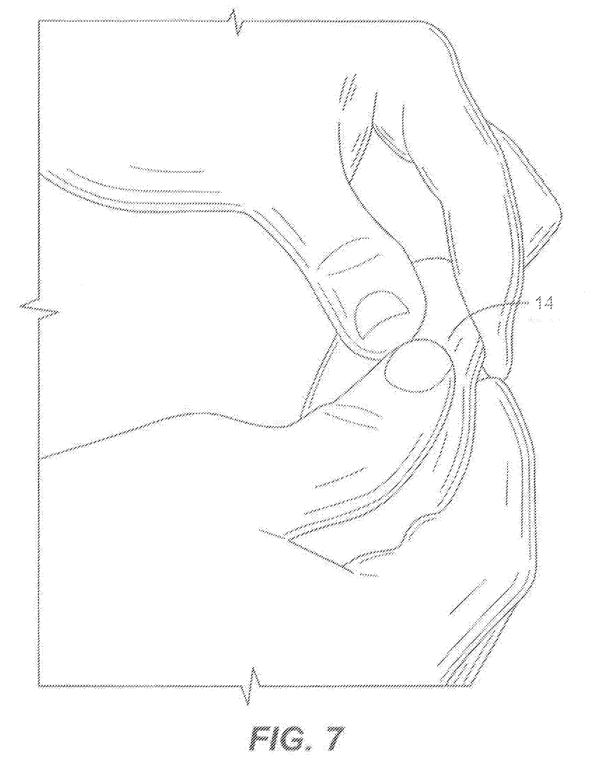

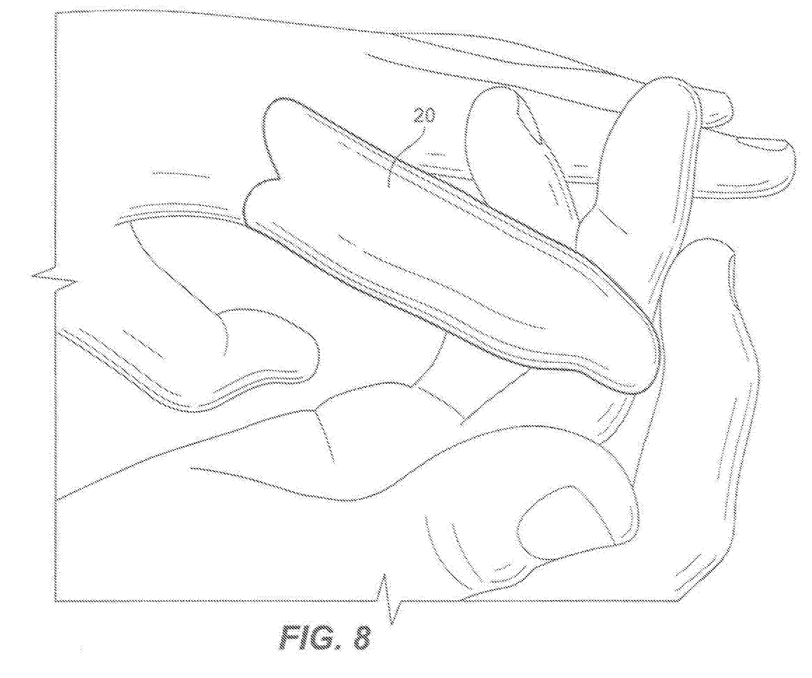

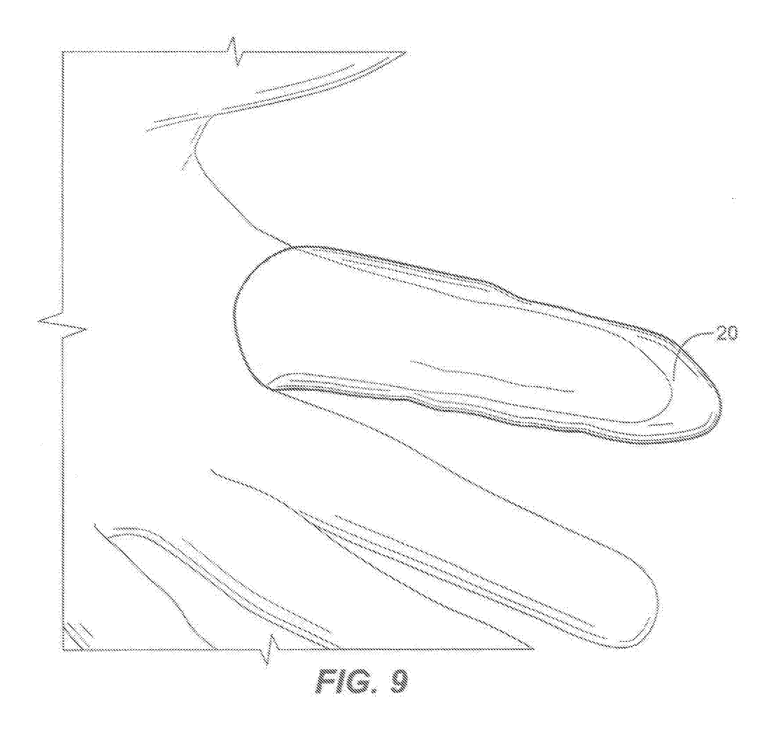

The malleable material is then shaped manually (FIG. 6) and then molded to any area F (FIG. 7), to form an injury protection device such as a splint 20 (FIGS. 8 and 9, step S112). Within minutes the splint 20 cools to room temperature and is shape stable and sets so that it is hard and rigid and protects the limb. Importantly, as the splint 20 cools, it returns to its solid and opaque appearance.

The malleable material can be used in many ways. For example, it can be stretched to form a wound protection device for an area where a catheter is placed on an animal's limb, providing support for the catheter, keeping it stable and allowing taping of the catheter to be accomplished over the catheter in such a way that the tape adheres to the wound protection device rather than adhering to the wound.

In step 114 a determination is made as to whether the splint 20 is sufficient (e,g., large enough to cover a desired portion of the limb, such as a joint) or whether splint 20 needs to be larger. If the splint 20 is large enough then the process is done. Optionally, the limb (e.g., finger F) FIG. 9 and splint 20 are wrapped with tape 18 in a conventional manner.

Certain heatable and formable materials, such as a material made of polycaprolactone can be formed and re-formed as well as added to an original formation with no apparent or significant loss in physical properties. So, if for some reason the splint 20 is not acceptable (for example, because it is too small), it is removed from the finger F, placed in an appropriate vessel (not shown, step S120) and the molded material is reheated using a microwave or any other means available (step 122). The softened material is then placed on the limb (finger F) FIG. 9 and remolded as necessary (step S124).

As shown in FIG. 9, if the splint is too small so that it does not cover a sufficient portion of the finger, then more formable material can be added to the splint. Alternatively, the entire splint may be re-heated and re-formed, incorporating additional, newly softened material as necessary. In step 114 if more material is required, another piece of the formable material is softened as described above, heated in step S 126 and may be added to the splint 20 (step 128). One piece of the formable material can be added readily to another piece that has been softened and formed into a splint previously.

The kit and method, which have been described above, allow for particularly malleable and thin material, to the point that it can be used for making a splint for the finger of a person. As can be seen in FIG. 8 or 9 (as examples), a physician, technician or user can position the injured finger. The material can serve as a bracing for the finger upon hardening. This approach is preferred to that of the current approach (a taped or plaster or fiberglass splint). Such splints are time consuming to construct and bulky alternatives. Such splints can be compromised if in contact with water.

The same method can be used to make other wound protection devices to cover and shield other portions of a person's body or to cover the portions the body of an animal. Similarly, the kit of the present invention can be used for other purposes including, for example, a means to form dental impressions.

Finally, while the invention has been described in conjunction with a kit and method for making a wound protection device for human or animal patients, similar kits could be made for mending elongated inanimate objects as well, such as broken electrical connectors.

In the description provided above, the protective sheet 16 is used to insure that the formable material 14 does not become contaminated by the heating composition. Other means may be used to achieve similar results. For example, the heating composition within the bag can be arranged so that it is separated from the formable material by an inner lining or by placing the heating composition in its own pouch, the lining or pouch being made of a water permeable sheet.

This kit could include easily understood instructions for making a wound protection device such as with or without an anti-microbial agent. The material may be placed directly over an area where there is a deficit of tissue for closing a wound. Used in this manner, the wound protection device may be sutured directly through it and sutured through the adjacent skin during the cooling process, forming a temporary protected area over the damaged tissue so healing may take place. Sometime later, when wound healing is sufficient, the wound protection device may be removed and closure may be accomplished. In another example, if a burn has injured the palm and fingers of a hand, the wound protection device may be directly molded over the injured area and between the fingers to at once protect the fingers and palm from additional injury, while separating the fingers.

Numerous modifications may be made to the invention without departing from its scope as defined in the appended claims.

1. A method for forming a mending device on a patient, customized for supporting or immobilizing a portion of the patient's body, comprising the steps of:
providing a mobile kit, said kit including:
a formable object malleable into an injury protection device at an elevated temperature suitable for placing on a human or animal patient, and retaining a rigid configuration at room temperature; and
a heating source actuatable absent a flame and used in proximity to said object to elevate said object's temperature;
heating the formable object to elevate its temperature;
molding said formable object into a required shape by placing and shaping said formable object on a portion of a patient; and
allowing said shaped object to cool to room temperature, thereby becoming rigid.

2. The method of claim 1, wherein said kit further includes a bag or other container for separating said object from said heating source.

3. The method of claim 1, wherein said kit further includes an actuator.

4. The method of claim 3, wherein said actuator includes a chemical composition.

5. The method of claim 3, wherein said actuator includes water.

6. The method of claim 3, wherein said activator includes an element of a hydrogen suppression system.

7. The method of claim 2, wherein said container further includes a flap which, when opened, allows air to enter and said air activates said heating source.

8. The method of claim 1 further comprising the step of wrapping said formable object with a protective sheet of particular qualities prior to said heating.

9. The method of claim 1, wherein said kit further includes an auxiliary forming object and said step of heating includes heating said auxiliary forming object.

10. The method of claim 1, wherein said formable object is sufficiently shape-stable at approximately 37±5° C.

11. The method of claim 1, wherein said formable object is suturable.

12. The method of claim 1 wherein said formable object is translucent.

13. The method of claim 1, wherein said patient is an animal.

14. The method of claim 1, wherein said formed injury protection device is usable to protect and separate fingers.

15. A kit for forming a device for protecting an injury in a patient comprising:
a formable object, said formable object having a rigid configuration at room temperature and being moldable at an elevated temperature;
an actuatable heating source; and
a separator for separating said heating source and said object;
wherein said heating source heats absent a flame.

16. The kit of claim 15, wherein said formable object is usable to protect and separate fingers.

17. The kit of claim 15, wherein said actuatable heating source comprises a hydrogen suppression system.

18. The kit of claim 15, wherein said separator is a protective sheet.

19. The kit of claim 15, wherein said separator is a bag.

20. The kit of claim 15, wherein said kit further includes an auxiliary forming object mergable with said formable object to create a larger protective device.
